(19) United States
(12) Patent Application Publication
(10) Pub. No.: US 2026/0272599 A1
(43) Pub. Date: Sep. 17, 2026

(54) ADJUSTABLE AND COMBINATION SURGICAL AIMER, DRILL GUIDE AND BULLET

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: João Pedro DE ALMEIDA BARRETO, Coimbra (PT); Rafal Z. JEZIERSKI, Candia, NH (US); Nathan ZAMARRIPA, Kittery Point, ME (US); Luis Carlos Fial TEIXEIRA RIBEIRO, Coimbra (PT); Geoffrey I. KARASIC, Raynham, MA (US); Philip A. CORMIER, Newburyport, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/648,286

(22) Filed: Apr. 15, 2026

Related U.S. Application Data
(63) Continuation-in-part of application No. PCT/US2024/052193, filed on Oct. 21, 2024.
(60) Provisional application No. 63/922,343, filed on Nov. 21, 2025.
(60) Provisional application No. 63/877,374, filed on Sep. 8, 2025.
(60) Provisional application No. 63/612,638, filed on Dec. 20, 2023.
(60) Provisional application No. 63/612,677, filed on Dec. 20, 2023.
(60) Provisional application No. 63/598,298, filed on Nov. 13, 2023.

(51) Int. Cl. A61B 90/11; A61M 25/09

(52) U.S. Cl. CPC A61B 90/11; A61M 25/09

(57) ABSTRACT

A surgical tool aimer can include a handle to support and aim a bullet that is configured to target a surgical site. An aimer arm can be coupled to the handle. The aimer arm can have a distal end with a tip and an elbow adjacent to the tip. The surgical tool aimer can aim the bullet at either the tip or the elbow.

### CROSS-REFERENCE

This application claims the benefit of U.S. Provisional Application No. 63/877,374 filed on September 8, 2025; and of U.S. Provisional Application No. 63/922,343 filed on November 21, 2025. This application is also a bypass continuation-in-part (CIP) of PCT/US2024/052193 filed on October 21, 2024 entitled ADJUSTABLE AND COMBINATION SURGICAL AIMER, DRILL GUIDE AND BULLET, which claims the benefit of U.S. Provisional Application No. 63/612,677 filed on December 20, 2023; U.S. Provisional Application No. 63/612,638 filed on December 20, 2023; and U.S. Provisional Application No. 63/598,298 filed on November 13, 2023. All of the noted applications are incorporated by reference herein as if reproduced in full below.

### BACKGROUND

### Field of the Disclosure

This disclosure relates in general to surgical navigation and, in particular, a system, method and apparatus for a surgical aimer that is adjustable and usable in combination with a drill guide and bullet.

### Description of the Prior Art

In computer-assisted arthroscopic surgery, fiducial blocks or fiducials with sensitive laser marks, such as passive reflective infrared fiducial markers, for example, can be mounted on an aimer and used to provide a coordinate system to localize instruments and anatomy. The optically detectable fiducials are affixed in a tracking array and attached to the aimer to allow the user to navigate their planned drill trajectories or scope of tissue resection, which can be done in conjunction with software on the camera control unit of the navigation system. To avoid damage to the fiducials, and to avoid cleaning and sterilization issues associated with the fiducials, single use disposable tracking arrays are used. Although conventional solutions are workable, improvements in aimers continue to be of interest.

### SUMMARY

Embodiments of a system, method and apparatus for a surgical tool aimer are disclosed. For example, the surgical tool aimer can include a handle to support and aim a bullet that is configured to target a surgical site. An aimer arm can be pivotally coupled to the handle. The aimer arm can have a distal end with a tip and an elbow adjacent to the tip. An actuator can be coupled to the handle for selectively indexing the aimer arm about a pivot, such that the tip or the elbow can be manually adjusted to be aimed at the bullet while the bullet and handle are stationary. Numerous other embodiments are disclosed herein.

### BRIEF DESCRIPTION OF THE DRAWINGS

Before one or more embodiments are described in detail, one skilled in the art will appreciate that they are not limited in its application to the details of construction, the arrangements of components, and the arrangement of steps set forth in the following detailed description or illustrated in the drawings and descriptions. Rather, they are capable of other embodiments and of being practiced or being carried out in various ways. Also, it is to be understood that the phraseology and terminology used herein is for the purpose of description and should not be regarded as limiting.

FIGS. 1 and 2 are side views of a first embodiment of a surgical tool aimer shown aimed at an elbow and a tip, respectively, of the aimer arm.

FIGS. 3 and 4 are partially transparent side views of FIGS. 1 and 2, respectively.

FIG. 5 is an enlarged, partially transparent isometric view of a handle of the surgical aimer arm of FIGS. 3 and 4.

FIG. 6 is a partially transparent side view of a second embodiment of a surgical tool aimer.

FIGS. 7 and 8 are side views of a third embodiment of a surgical tool aimer shown aimed at a tip and elbow, respectively, of the aimer arm.

FIG. 9 is a side view of a fourth embodiment of a surgical tool aimer.

FIGS. 10 and 11 are side and isometric views, respectively, of an embodiment of a spring clip employed by the tool of FIG. 9.

FIGS. 12 and 13 are side and rear views, respectively, of a fifth embodiment of a surgical tool aimer.

FIG. 14 is an enlarged, partially transparent isometric view of a handle of the surgical aimer arm of FIGS. 12 and 13.

FIG. 15 is an isometric view of an alternate embodiment of a spring clip employed by the tool of FIG. 14.

FIGS. 16 and 17 are rear and exploded isometric views, respectively, of a sixth embodiment of a surgical tool aimer.

FIG. 18 is a rear view of a seventh embodiment of a surgical tool aimer related to the tool of FIGS. 20 and 21.

FIG. 19 is a schematic, isometric view of another embodiment of a surgical tool aimer depicting aim angles.

FIGS. 20 and 21 are side and isometric views, respectively, of another embodiment of a surgical tool aimer.

FIGS. 22 and 23 are side and sectional views, respectively, of another embodiment of a surgical tool aimer.

FIG. 24 is an isometric view of an aimer arm for the tool of FIGS. 22-23.

FIGS. 25 and 26 are isometric and side views, respectively, of another embodiment of a surgical tool aimer.

FIGS. 27 and 28 are isometric views of still another embodiment of a surgical tool aimer.

FIG. 29 is another isometric view of the embodiment of FIGS. 27 and 28, with a portion of the surgical tool aimer shown in phantom lines to illustrate an interior thereof.

FIG. 30 is a schematic, isometric view of an embodiment of a surgical tool aimer depicting aim angles.

FIG. 31 is a schematic, isometric view of another embodiment of a surgical tool aimer depicting aim angles.

### DETAILED DESCRIPTION

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings.

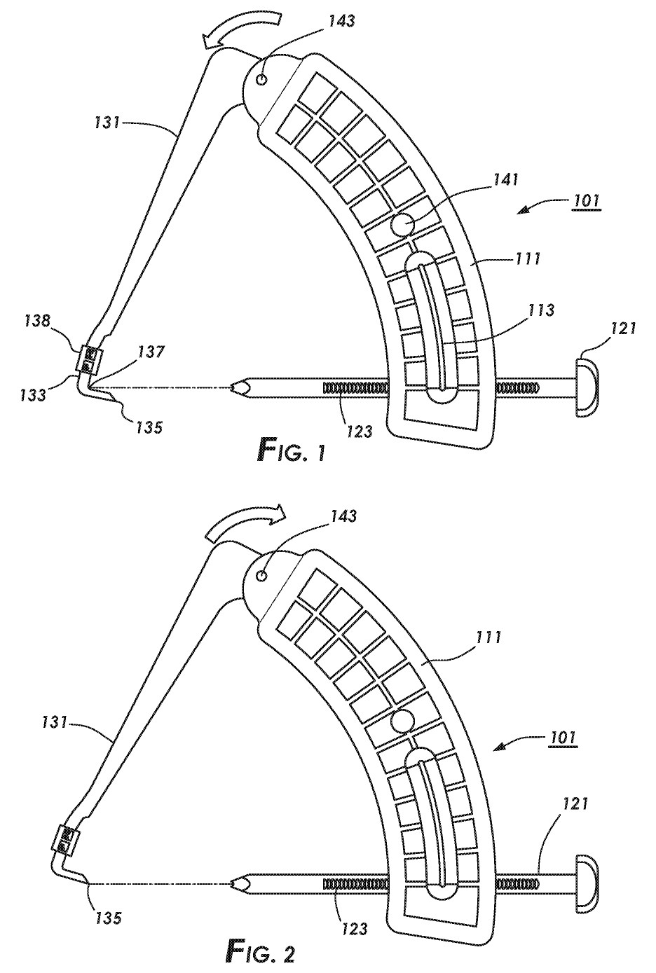

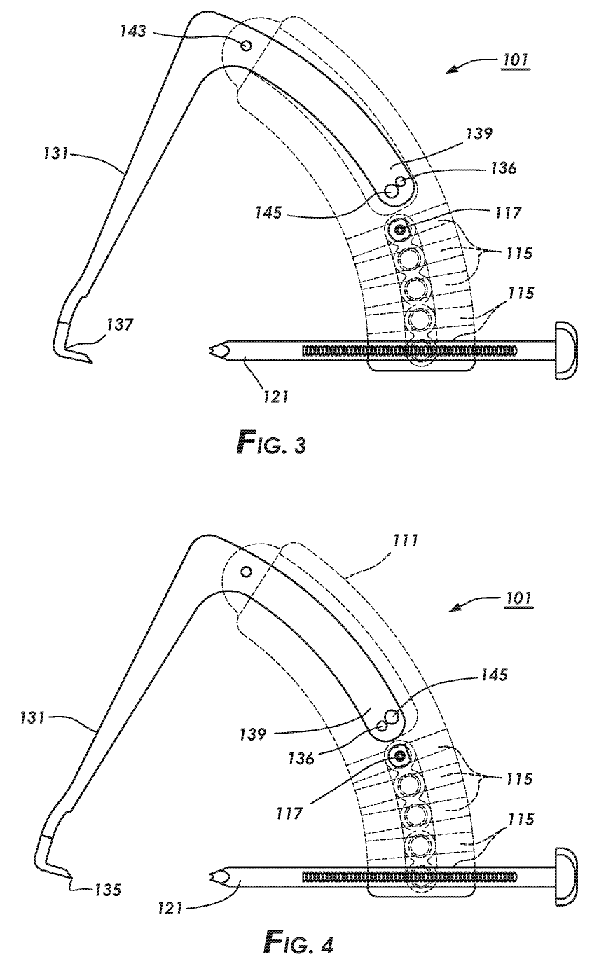

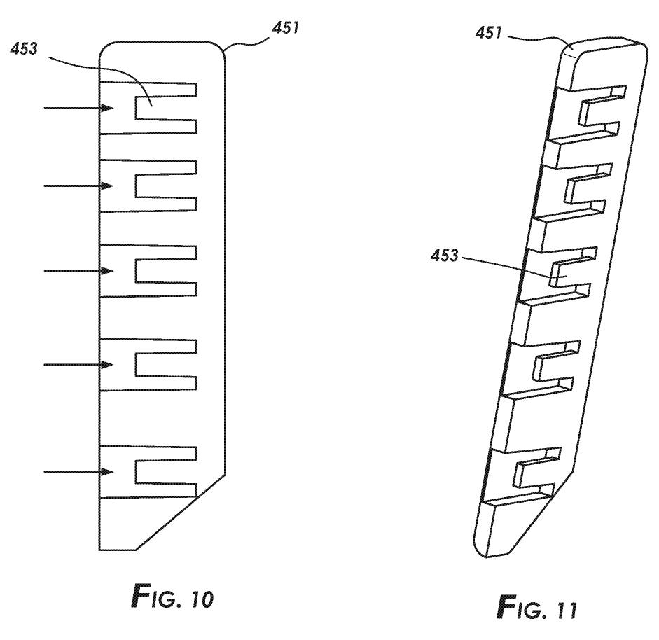

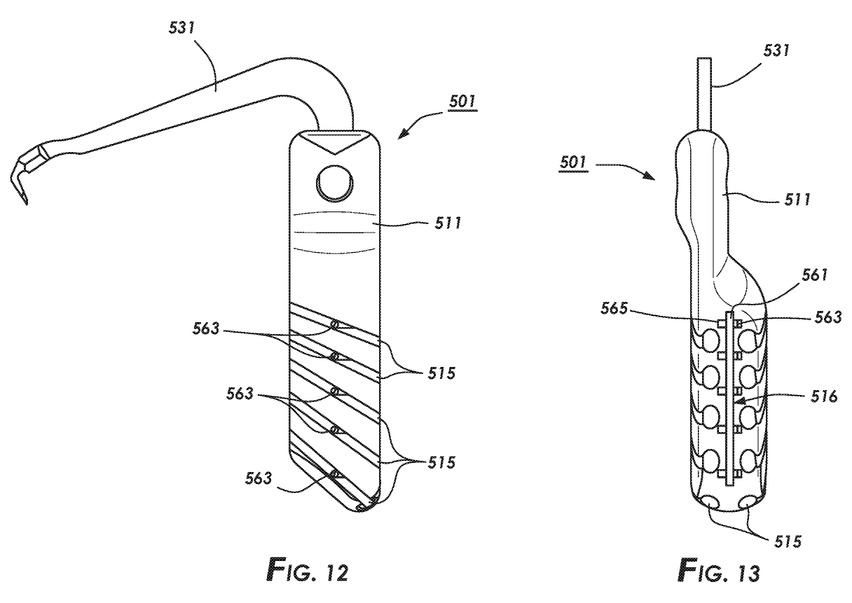

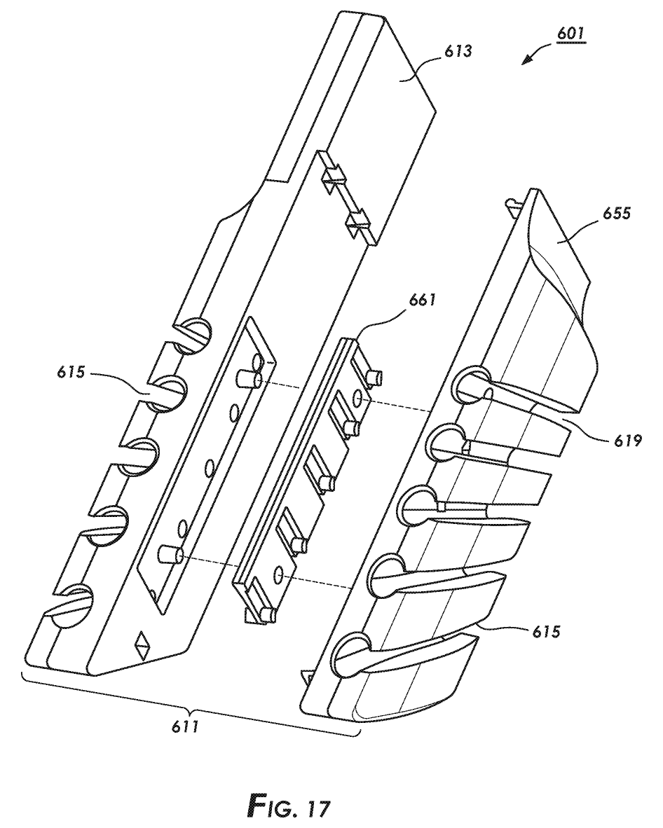

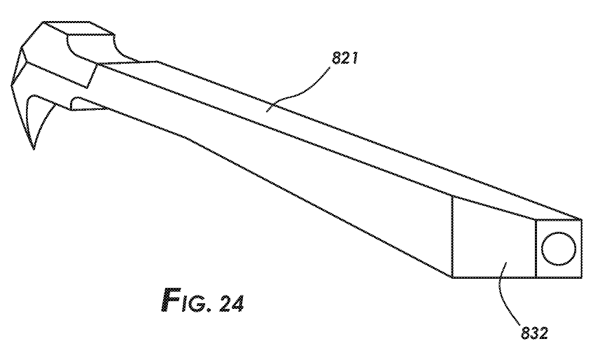

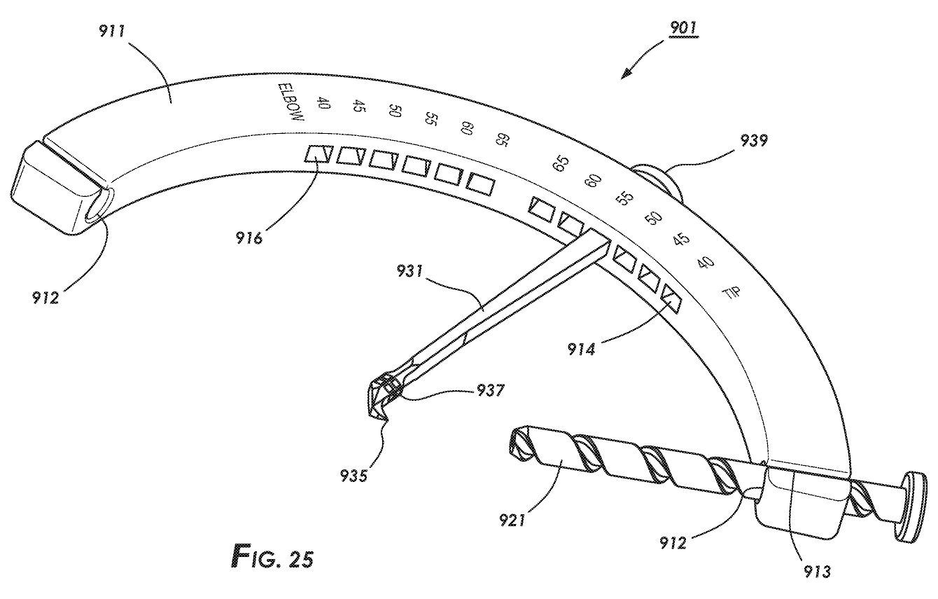

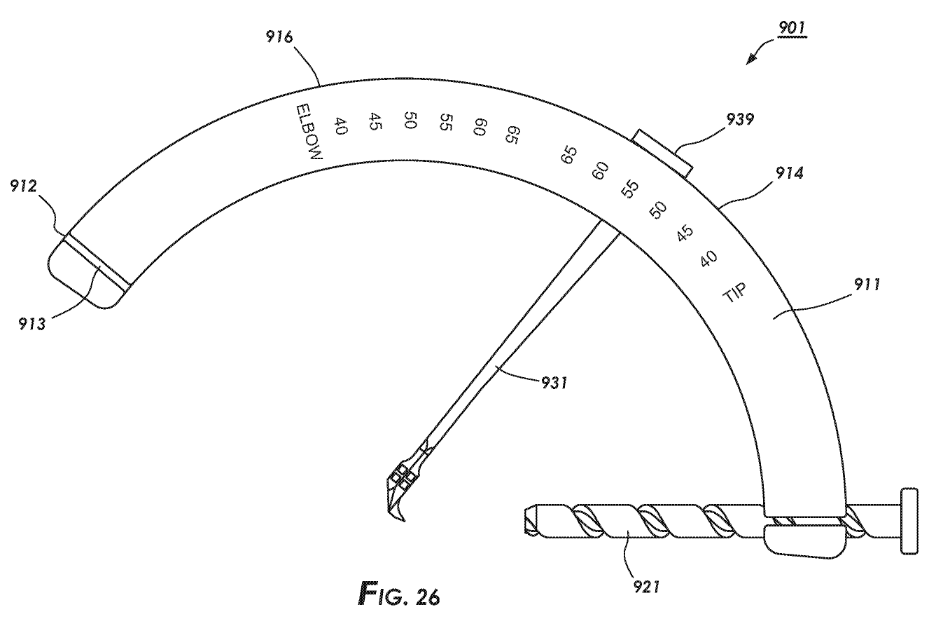

Referring to FIGS. 1-31, embodiments of a surgical tool aimer are disclosed. For example, FIGS. 1-6 disclose a surgical tool aimer 101 having a handle 111 that is configured to support and aim a bullet 121 that is configured to target a surgical site. In the embodiment shown, the handle 111 includes an aperture or side window 113 for access to the various positions of the bullet 121. In some versions, the handle 111 does not comprise any side windows for the bullet 121.

An aimer arm 131 can be pivotally coupled to the handle 111. The aimer arm 131 can include a distal end 133 with a tip 135 and an elbow 137 adjacent to the tip 135. An actuator 141 can be coupled to the handle 111 for selectively indexing the aimer arm 131 about a pivot 143. Pivotally moving the aimer arm 131 allows the tip 135 or the elbow 137 to be manually adjusted to be aimed at the bullet 121 while the bullet 121 and the handle 111 are stationary. As shown in FIGS. 1-4, the pivot 143 can be external relative to the handle 111.

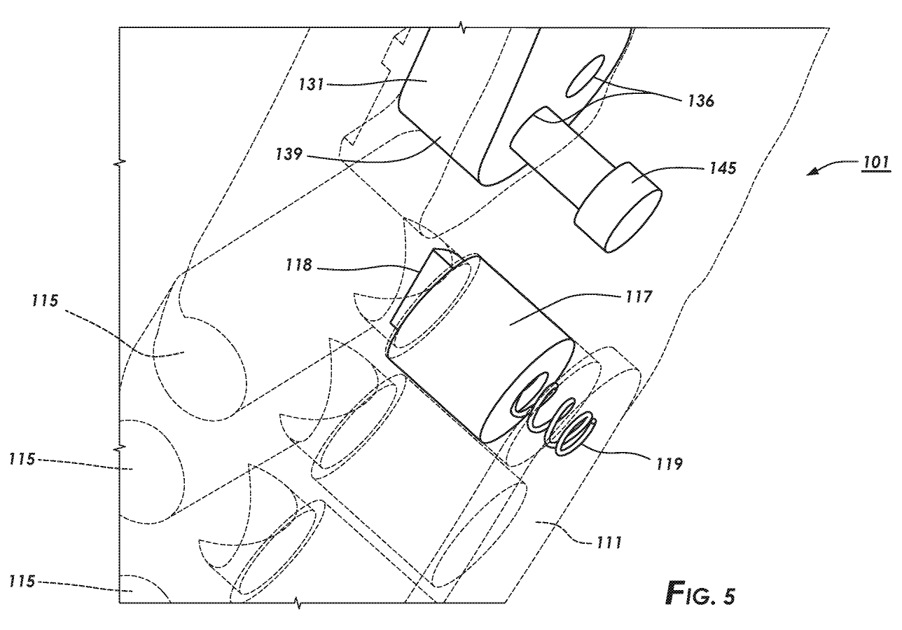

In some embodiments (FIGS. 3-5), the actuator 141 comprises a pin 145 that is manually actuated by a user of the surgical tool aimer 101. The pin 145 can be manually moved to engage and retain the aimer arm 131 in a desired position. The proximal end 139 of the aimer arm 131 can include holes 136 that can selectively receive the pin 145 (when moved manually) to secure the aimer arm 131 in desired position.

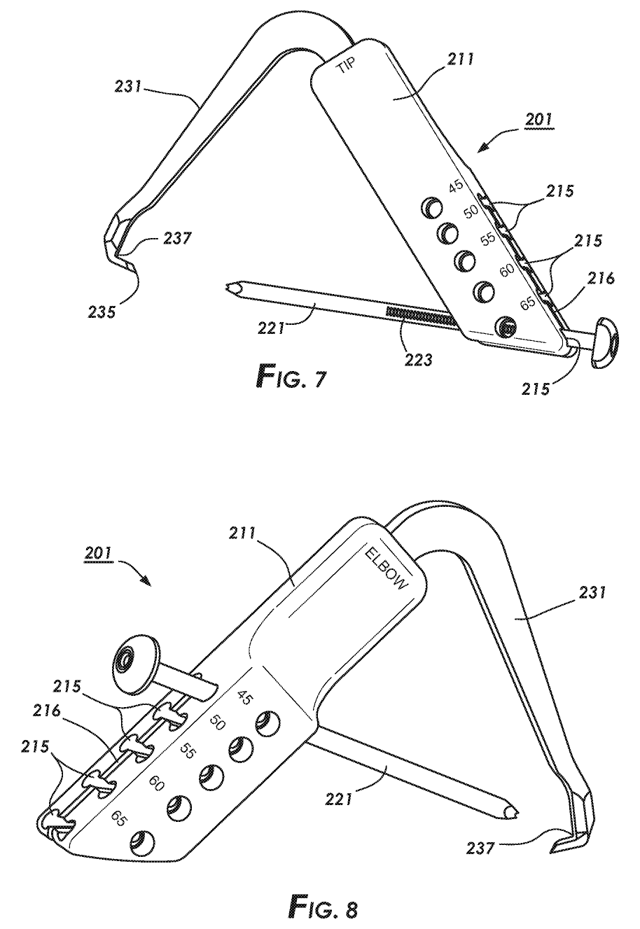

In other embodiments (FIG. 6), the actuator 141 comprises magnets 147 located on opposite sides of the interior of the handle 111, as shown. The magnets 147 can selectively engage a proximal end 139 of the aimer arm 131, opposite the tip 135 and elbow 137 of the aimer arm 131, in indexed positions when the aimer arm 131 is manually pivoted about pivot 143. In some versions, the pivot 143 can be inside the handle 111. The proximal end 139 of the aimer arm 131 can extend into an interior of the handle 111 for about half of a length of the handle 111. The handle 111 can be curved rather than straight.

Embodiments of the bullet 121 and the aimer arm 131 can be in co-planar alignment. For example, the longitudinal centerlines of the bullet 121 and the aimer arm 131 can be in co-planar alignment. The aimer arm 131 can include a fiducial marker 138 that is configured to assist in video-based surgical navigation.

The handle 111 can include receptacles 115 for the bullet 121. The bullet 121 is configured to be installed in any one of the receptacles 115 to selectively vary an angle of inclination of the bullet 121 toward the surgical site. The receptacles 115 can comprise a single column of receptacles 115. The bullet 121 can be co-planar with the aimer arm 131.

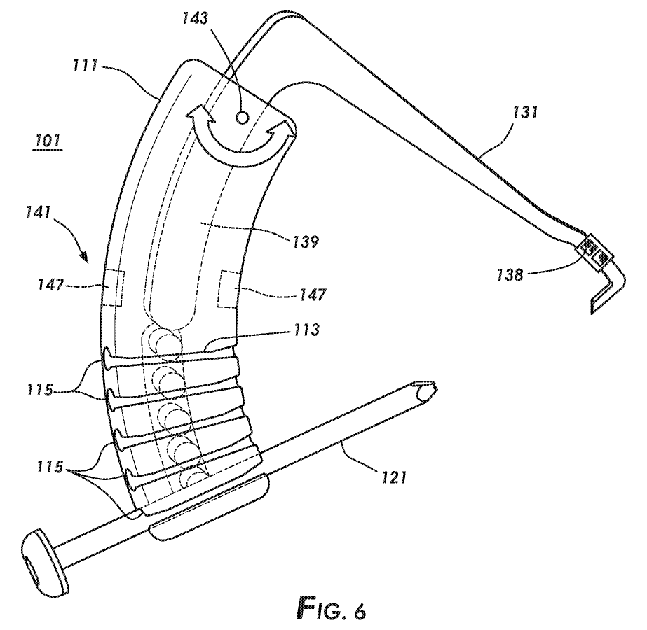

In some embodiments (FIGS. 3-5), a plunger 117 (only one shown, for ease of illustration) can be located adjacent to each receptacle 115 inside the handle 111. Each plunger 117 is located in a seat and biased toward a respective receptacle by a spring 119. Each plunger 117 has a tab 118 (FIG. 5) for selectively engaging teeth 123 on the bullet 121 to retain the bullet 121 in a respective receptacle 115. This can occur in a ratcheting style that is analogous to a caulk gun.

The aimer arm 131 can be disposable and the handle 111 can be reusable. For example, the aimer arm 131 can be metal and laser marked (single piece) and can be attached to the handle 111 at multiple angles. Alternatively, the surgical aimer tool 101 can be fully disposable with the same metal laser-marked aimer arm 131 and a plastic handle 111 and bullet 121. Multiple lasers marked metal aimer arms 131 can be provided to allow different shapes/functions like tip/elbow or others along with a single handle 111 and bullet 121. To reduce the number of components, the bullet 121 can be threaded with a big pitch to enable quick insertion/removal and allowing for fine tuning of the bullet position.

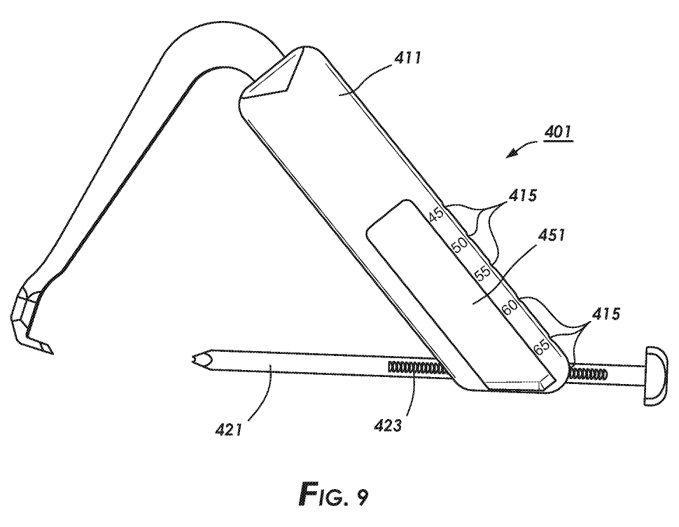

FIGS. 7-19 disclose other embodiments of surgical tool aimers. For example, a surgical tool aimer 201 (FIGS. 7 and 8) can include a handle 211 to support and aim a bullet 221 that is configured to target a surgical site. The handle 211 can have receptacles 215 for the bullet 221. Each of the receptacles 215 can be oriented at a unique orientation relative to the handle 211.

An aimer arm 231 can be rigidly mounted to the handle 211 such that there is no relative motion therebetween. The aimer arm 231 can include a tip 235 and an elbow 237 adjacent to the tip 235. The handle 211 can be configured to retain the bullet 221 in one of the receptacles 215 for selectively aiming the bullet 221 at either the tip 235 or the elbow 237. The aimer arm 231 and the bullet 221 can be laterally offset from a centerline of the handle 211. Versions of the aimer arm 231 and bullet 221 are not co-planar, such that the bullet 221 is skewed relative to the aimer arm 231.

In some versions (FIG. 18), a surgical tool aimer 301 can have a handle 311 with receptacles 315 that comprise a single column of receptacles 315. The bullet (when installed in handle 311) is not co-planar with the aimer arm.

In some embodiments (e.g., FIGS. 7-8), the receptacles 215 comprise two columns of receptacles 215. When installed, some versions of the bullet 221 are not co-planar with the aimer arm 231, such that the bullet 221 is offset and skewed relative to the aimer arm 231. As shown in FIG. 7, a first column (left side) of the two columns of receptacles 215 aims the bullet 221 at the tip 235 of the aimer arm 231, and a second column (FIG. 8, right side) of the two columns of receptacles 215 aims the bullet 221 at the elbow 237 of the aimer arm 231. A slot 216 can be located in the handle 211 between the two columns of receptacles 215.

Each receptacle 215 can comprise ratchet features configured to selectively engage and retain the bullet 221. The ratchet features can engage teeth 223 on the bullet 221. For example, in FIGS. 9-11, a surgical tool aimer 401 has a handle 411 that comprises opposed spring clips 451 that include the ratchet features. Each spring clip 451 has a tooth 453 for each respective receptacle 415 in the handle 411. The teeth 453 are configured to engage the teeth 423 of the bullet 421 and retain the bullet 421 in the respective receptacles 415.

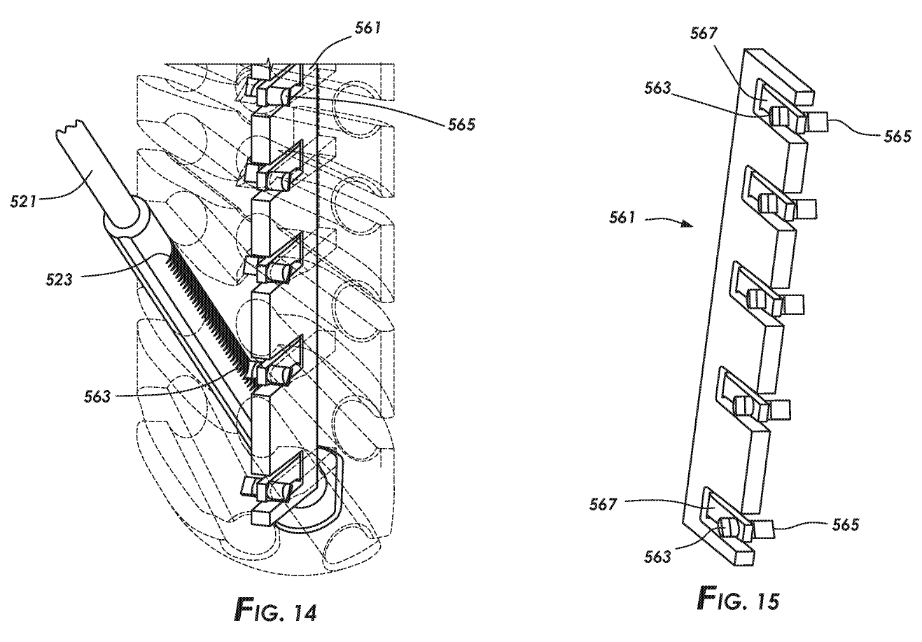

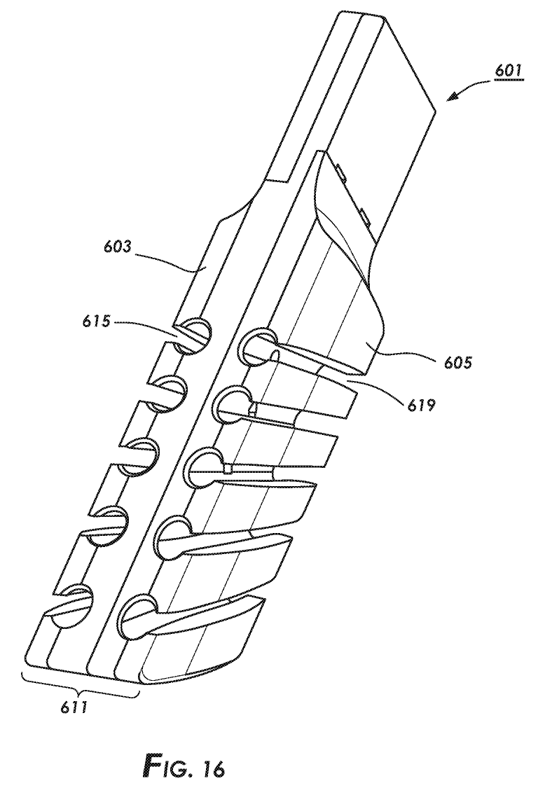

As shown in FIGS. 12-15, embodiments of a surgical tool aimer 501 can include a lock plate 561 comprising the ratchet features. The lock plate 561 can be located inside the handle 511. The lock plate 561 can have a tooth (e.g., two types of teeth 563, 565 are shown) for each respective receptacle 515. The lock plate 561 can be located in a slot 516 (FIG. 13) in the handle 511 that is between columns of the receptacles 515, such that the lock plate 561 is mounted in the slot 516.

Embodiments of the lock plate 561 can be asymmetric. For example, the teeth 563 for a first column of the receptacles 515 can be smaller than the teeth 565 for a second column of the receptacles 515. Pairs of the teeth 563, 565 can extend from respective tabs 567. Each tab 567 can be cantilevered from the lock plate 561. Each pair of the teeth 563, 565 can be perpendicular to the respective tab 567.

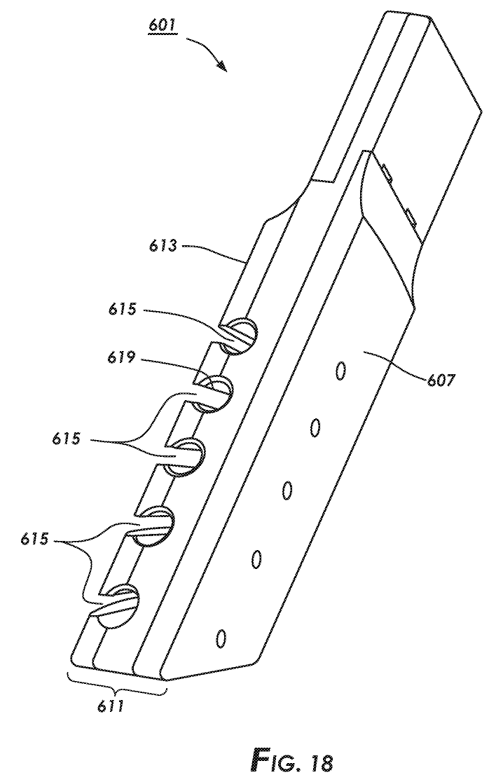

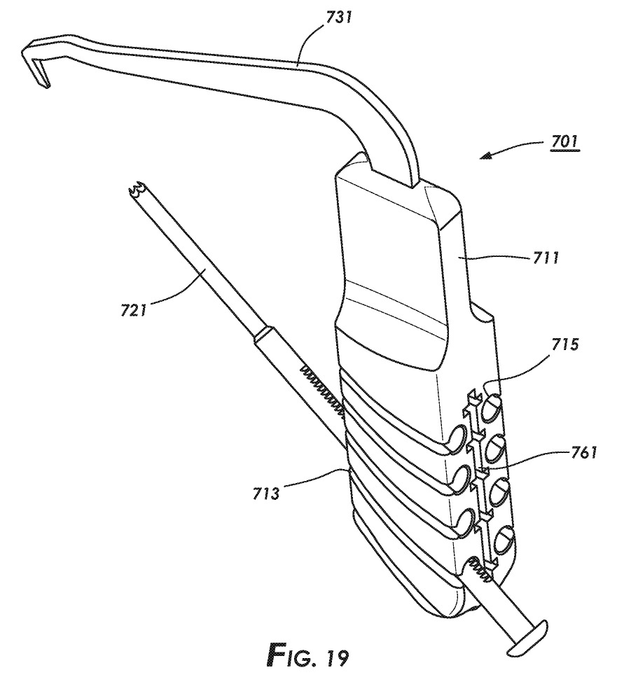

FIGS. 16-18 depict embodiments of a surgical tool aimer 601 that is reconfigurable. For example, in FIGS. 16 and 17, the handle 611 comprises a primary handle 603 and a secondary handle 655, each with receptacles 615 for a bullet (not shown), that can be assembled to retain a lock plate 661. The lock plate 661 is described elsewhere herein. Each receptacle 615 can have a side window 619 to an exterior of the handle 611. Alternatively, the secondary handle 655 can be replaced by an alternate handle 607 (FIG. 18) that does not have any receptacles 615, but still retains the lock plate 661 inside the handle 611.

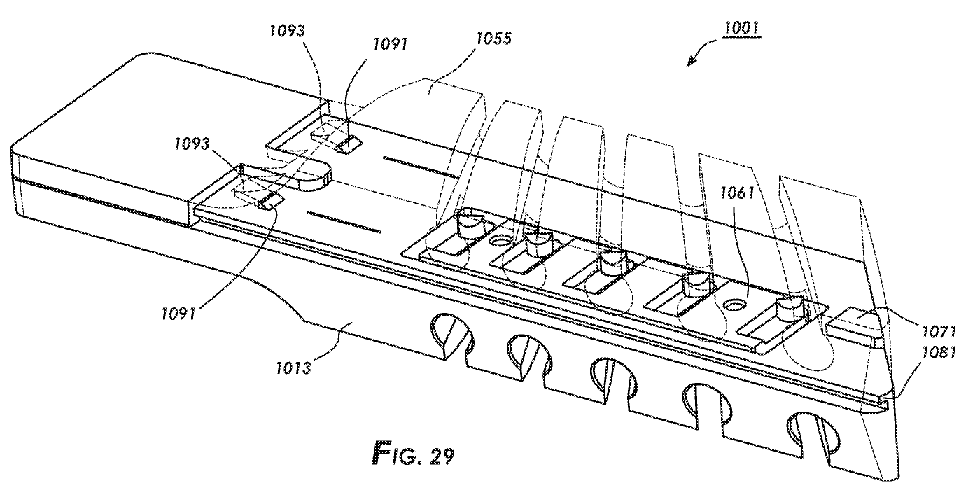

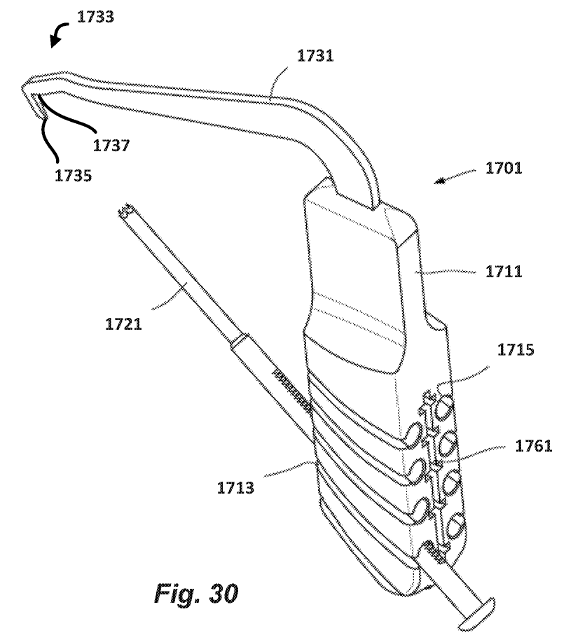

In FIGS. 27-29, a variation of the preceding embodiment is shown. In this version, a surgical tool aimer 1001 also is reconfigurable. The handle 1011 comprises a primary handle 1013 and a secondary handle 1055, each with receptacles 1015 for a bullet (not shown), that can be assembled to retain a lock plate 1061 (FIG. 29). The lock plate 1061 is described elsewhere herein. Each receptacle 1015 can have a side window 1019 to an exterior of the handle 1011. The secondary handle 1055 slidably and, in some cases, releasably engages the primary handle 1013 to retain the lock plate 1061 inside the handle 1011. Several retention features are used to align and secure the secondary handle 1055 to the primary handle 1013. These include a central rib 1071 on the primary handle 1013 that engages a central slot 1073 on the secondary handle 1055. As shown in FIG. 28, there also are flanges 1081 on opposite sides of the perimeter of the primary handle 1013 that slidably engage complementary flanges 1083, respectively, on opposite sides of the perimeter of the secondary handle 1055. Finally, the primary handle 1013 has tabs 1091 (FIG. 29) that engage spring tabs 1093 on the secondary handle 1055. In this example, the secondary handle 1055 is not intended to be releasable from primary handle 1013, such that their assembly is permanent.

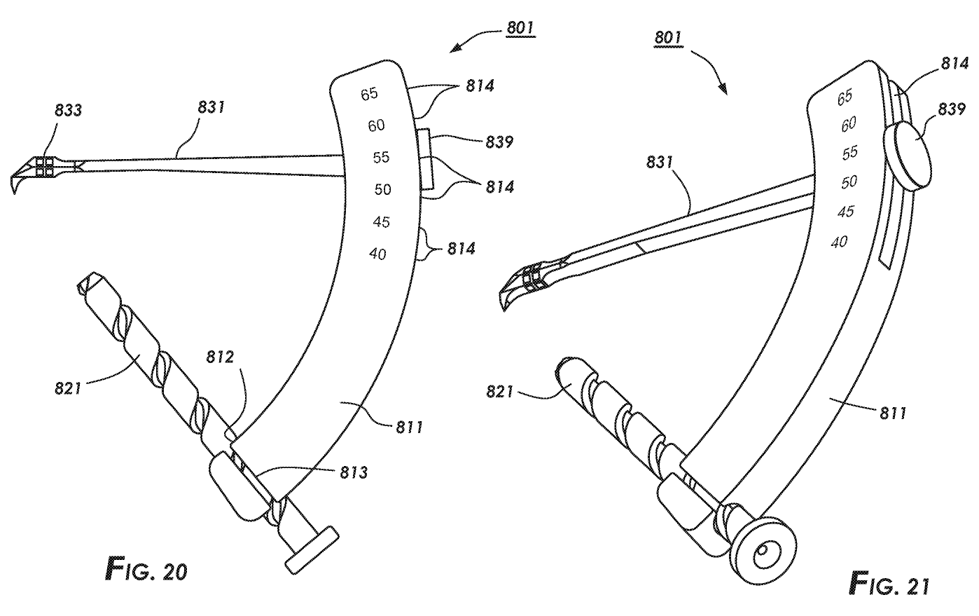

FIG. 19 depicts another embodiment of a surgical tool aimer 701. A handle 711 supports and aims a bullet 721 to target a surgical site. The handle 711 has receptacles 715 for the bullet 721. Each of the receptacles 715 is oriented at a unique orientation relative to the handle 711. The handle 711 is symmetrical about a longitudinal centerline.

An aimer arm 731 can be rigidly mounted and not movable relative to the handle 711. The aimer arm 731 has a tip 735 and an elbow 737 adjacent to the tip 735. The handle 711 retains the bullet 721 in one of the receptacles 715 to selectively aim the bullet 721 at either the tip 735 or the elbow 737. The aimer arm 731 can be co-planar with a centerline of the handle 711.

The receptacles 715 can comprise first and second columns of receptacles 715. The first column of the receptacles 715 can aim the bullet at the tip 735, and the second column of the receptacles 715 can aim the bullet at the elbow 737. A lock plate 761, as described elsewhere herein, can be located inside the handle 711. The lock plate 761 can be symmetric or asymmetric.

In some embodiments, the bullet 721 is not co-planar with the aimer arm 731 such that the bullet 721 is skewed relative to the aimer arm 731. The handle 711 can comprises a longitudinal slot 713 (e.g., side window) outboard of each receptacle 715. The longitudinal slots 713 can enable guidewire removal from respective ones of the receptacles 715.

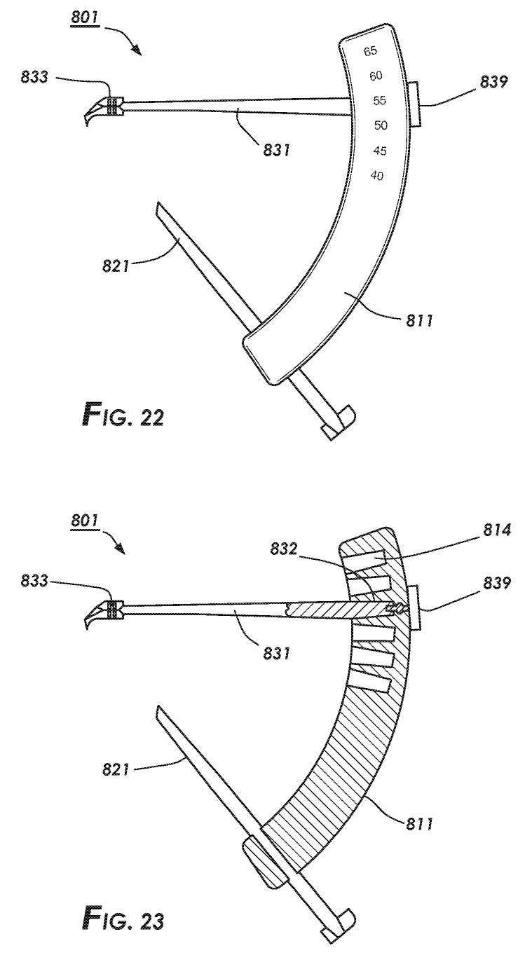

Unlike the other embodiments, FIGS. 20-26 disclose a surgical aimer tool with a repositionable aimer arm and a fixed bullet. For example, the surgical aimer tool 801 (FIGS. 20-24) can include a handle 811 with only one bullet receptacle 812 (with an optional side window 813) for supporting a bullet 821. The bullet receptacle 812 can be the only location on the handle 811 to which the bullet 821 can be mounted. The handle 811 can aim the bullet 821 at target a surgical site.

Versions of the handle 811 can include a plurality of aimer receptacles 814. An aimer arm 831 can comprise metal and a laser-marked fiducial 833 configured to assist in video-based surgical navigation. The aimer arm 831 comprises a tip 835 and an elbow 817 adjacent to the tip 835. The aimer arm 831 can be installed in any one of the aimer receptacles 814 to selectively vary an angle of inclination of the aimer arm 831 relative to the handle 811 and the bullet 821. In some versions, the bullet 821 can be selectively aimed at either the tip 835 or the elbow 837 of the aimer arm 831.

In one version, the handle 811 can be reusable and the aimer arm 831 can be disposable. In another version, both the handle 811 and the aimer arm 831 are disposable. The bullet 821 can be threaded into the handle 811 to adjust a length of the bullet 821 relative to the aimer arm 831. The handle 811 can be arcuate in shape and the aimer arm 831 and bullet 821 can be linear in shape, other than a distal tip of the aimer arm 831.

A proximal end 832 of the aimer arm 831 can be tapered, as shown. Each of the aimer receptacles 814 are complementary in shape to the proximal end 832 of the aimer arm 831. Examples of the handle 811, bullet 821 and aimer arm 831 can be in co-planar alignment. Longitudinal centerlines of the handle 811, bullet 821 and aimer arm 831 can be in co-planar alignment, in some versions.

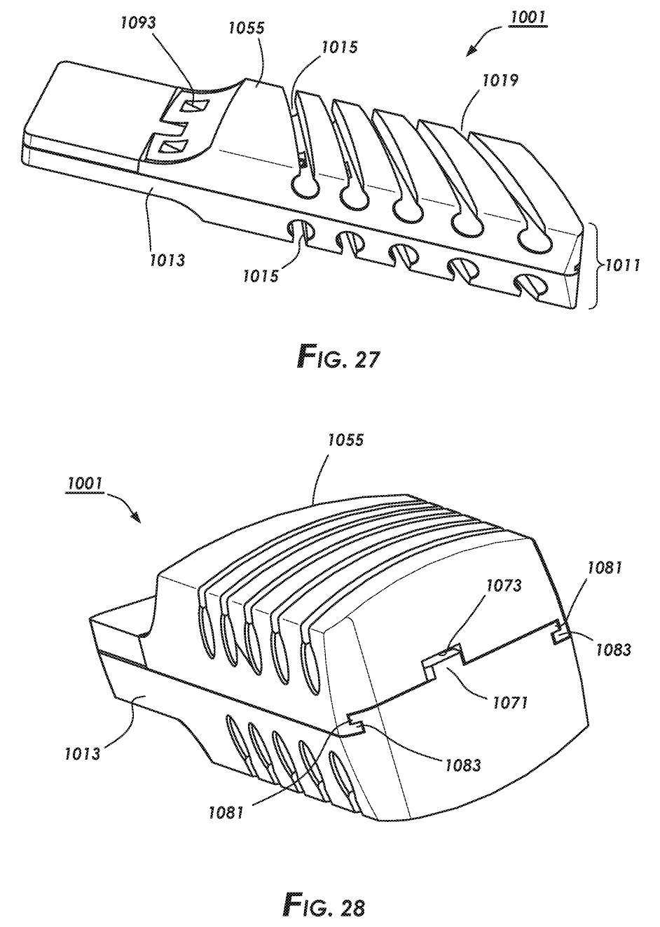

As shown in FIGS. 25-26, another embodiment of a surgical tool aimer 901 can have aimer receptacles that comprise a first array of aimer receptacles 914 and a second array of aimer receptacles 916. This version also can have two bullet receptacles 912. The handle 911 can include a side window 913 for the bullet 921. Each aimer receptacle of the first array of aimer receptacles 914 is configured to aim the bullet 921 only at the tip 935. Each aimer receptacle of the second array of aimer receptacles 916 is configured to aim the bullet 921 only at the elbow 937. The first array of aimer receptacles 914 can be located adjacent a proximal end of the handle 911, and the second array of aimer receptacles 916 can be located adjacent a distal end of the handle 911.

Embodiments of the aimer arm 931 can be mounted in any one of the aimer arm receptacles 914, 916. Otherwise, the aimer arm 931 is not otherwise adjustable relative to the handle 911, in some versions. The aimer arm 931 can extend through a front of the handle 911, through one of the aimer receptacles 914, 916. The aimer arm 931 can be releasably secured to the handle 911 with a fastener 939 located on a rear of the handle 911.

It may be additionally useful to provide an aimer to guide trajectory of a guidewire by selective placement of a bullet into receptacles on either side of a handle of the aimer that all cause the bullet to aim towards the same location along the aimer arm, such as all aiming towards the tip, or all aiming towards the elbow, or aiming to some other single target location along the aimer arm, rather than (for example) requiring one column of the receptacles to aim towards the tip while the other column of the receptacles of the same aimer are aiming towards the elbow. In this manner, the user of the aimer could choose an appropriate receptacle on either side of the handle with a view to avoiding wedging of the handle between guidewires, particularly during a meniscal root repair as part of ACL reconstruction that may require multiple guidewires, while still being able to aim the bullet at the same target location.

FIG. 30 depicts an embodiment of a surgical tool aimer 1701. An aimer arm 1731 includes a distal end 1733 with a tip 1735 and an elbow 1737 adjacent to the tip 1735. The aimer arm 1731 is rigidly mounted to a handle 1711 that itself supports and aims a bullet 1721 to target a surgical site. The handle 1711 has receptacles 1715 for the bullet 1721. The receptacles 1715 comprise first and second columns of receptacles 1715. The receptacles 1715 of the first column of the receptacles 1715 aim the bullet at a single target location along the aimer arm 1731, and the receptacles 1715 of the second column of receptacles 1715 aim the bullet at the same single target location along the aimer arm 1731. In this particular example, the receptacles 1715 of the first column of the receptacles 1715 aim the bullet at the tip 1735, and the receptacles 1715 of the second column of the receptacles 1715 also aim the bullet at the tip 1735. Each of the receptacles 1715 in the first column of receptacles 1715 is oriented at a unique orientation relative to the handle 1711, and each of the receptacles 1715 in the second column of receptacles 1715 mirrors (or “matches”), about the longitudinal centerline of the handle 1711, the orientation of a respective receptacle 1715 in the first column of receptacles 1715. Therefore, for a given trajectory of bullet 1721, the user of the surgical tool aimer 1701 may choose to have the bullet 1721 received within a receptacle 1715 of the first column or to have the bullet 1721 received within the receptacle 1715 of the second column that is just across the longitudinal centerline of the handle 1711, depending on which the user feels would be more appropriate for avoiding wedging of the aimer 1701 while providing the corresponding selected orientation of bullet 1721. The handle 1711 is symmetrical about a longitudinal centerline.

The aimer arm 1731 is not movable relative to the handle 1711. The handle 1711 retains the bullet 1721 in one of the receptacles 1715 to selectively orient the bullet 1721 to aim the bullet 1721 at the tip 1735. The aimer arm 1731 can be co-planar with a centerline of the handle 1711.

In this embodiment, the bullet 1721 is not co-planar with the aimer arm 1731 such that the bullet 1721 is skewed relative to the aimer arm 1731. The handle 1711 can comprises a longitudinal slot 1713 (e.g., side window) outboard of each receptacle 1715. The longitudinal slots 1713 can to enable guidewire removal from respective ones of the receptacles 1715.

A slot 1761 can be located in the handle 1711 between the two columns of receptacles 1715.

Each receptacle 1715 can comprise ratchet features configured to selectively engage and retain the bullet 1721. The ratchet features can engage teeth on the bullet 1721. For example, the surgical tool aimer 1701 may have a handle 1711 that comprises opposed spring clips that include the ratchet features. Each spring clip may have a tooth for each respective receptacle 1715 in the handle 1711. The teeth may be configured to engage the teeth of the bullet 1721 and retain the bullet 1721 in the respective receptacles 1715.

Embodiments of a surgical tool aimer 1701 can include a lock plate comprising the ratchet features. The lock plate can be located inside the handle 1711. The lock plate can have a tooth for each respective receptacle 1715. The lock plate can be located in the slot 1761 in the handle 1711 that is between columns of the receptacles 1715, such that the lock plate is mounted in the slot 1761.

Embodiments of the lock plate can be asymmetric. For example, the teeth for a first column of the receptacles 1715 can be smaller than the teeth for a second column of the receptacles 1715. Pairs of the teeth can extend from respective tabs. Each tab can be cantilevered from the lock plate. Each pair of the teeth can be perpendicular to the respective tab.

In another embodiment, rather than all receptacles in both columns aiming the bullet towards the tip, all receptacles in both columns may aim the bullet towards another single target location along the aimer arm 1733, such as the elbow 1737, or some other point along the aimer arm 1733.

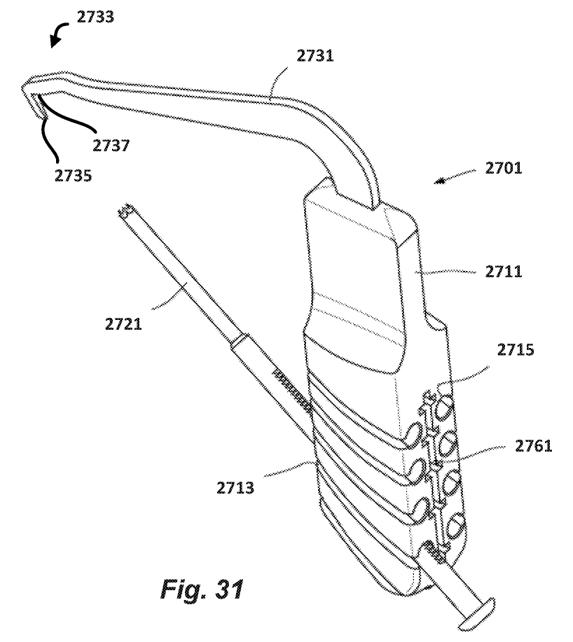

For example, FIG. 31 depicts an embodiment of a surgical tool aimer 2701. An aimer arm 2731 includes a distal end 2733 with a tip 2735 and an elbow 2737 adjacent to the tip 2735. The aimer arm 2731 is rigidly mounted to a handle 2711 that itself supports and aims a bullet 2721 to target a surgical site. The handle 2711 has receptacles 2715 for the bullet 2721. The receptacles 2715 comprise first and second columns of receptacles 2715. The receptacles 2715 of the first column of the receptacles 2715 aim the bullet at a single target location along the aimer arm 2731, and the receptacles 2715 of the second column of receptacles 2715 aim the bullet at the same single target location along the aimer arm 2731. In this particular example, the receptacles 2715 of the first column of the receptacles 2715 aim the bullet at the elbow 2737, and the receptacles 2715 of the second column of the receptacles 2715 also aim the bullet at the elbow 2737.

It will be appreciated that the aimers 1701 and 2701 appear, as depicted in FIGS. 30 and 31, to be identical, but it is intended that they be recognized as differing from each other in that all receptacles 1715 of the aimer 1701 of FIG. 30 aim the bullet 1721 at the tip 1735, and all receptacles 2715 of the aimer 2701 of FIG. 31 aim the bullet 2721 at the elbow 2737.

Returning to FIG. 31, each of the receptacles 2715 in the first column of receptacles 2715 is oriented at a unique orientation relative to the handle 2711, and each of the receptacles 2715 in the second column of receptacles 2715 mirrors (or “matches”), about the longitudinal centerline of the handle 2711, the orientation of a respective receptacle 2715 in the first column of receptacles 2715. Therefore, for a given trajectory of bullet 2721, the user of the surgical tool aimer 2701 may choose to have the bullet 2721 received within a receptacle 2715 of the first column or to have the bullet 2721 received within the receptacle 2715 of the second column that is just across the longitudinal centerline of the handle 2711, depending on which the user feels would be more appropriate for avoiding wedging of the aimer 2701 while providing the corresponding selected orientation of bullet 2721. The handle 2711 is symmetrical about a longitudinal centerline.

The aimer arm 2731 is not movable relative to the handle 2711. The handle 2711 retains the bullet 2721 in one of the receptacles 2715 to selectively orient the bullet 2721 to aim the bullet 2721 at the elbow 2737. The aimer arm 2731 can be co-planar with a centerline of the handle 2711.

In this embodiment, the bullet 2721 is not co-planar with the aimer arm 2731 such that the bullet 2721 is skewed relative to the aimer arm 2731. The handle 2711 can comprises a longitudinal slot 2713 (e.g., side window) outboard of each receptacle 2715. The longitudinal slots 2713 can to enable guidewire removal from respective ones of the receptacles 2715.

A slot 2761 can be located in the handle 2711 between the two columns of receptacles 2715.

Each receptacle 2715 can comprise ratchet features configured to selectively engage and retain the bullet 2721. The ratchet features can engage teeth on the bullet 2721. For example, the surgical tool aimer 2701 may have a handle 2711 that comprises opposed spring clips that include the ratchet features. Each spring clip may have a tooth for each respective receptacle 2715 in the handle 2711. The teeth may be configured to engage the teeth of the bullet 2721 and retain the bullet 2721 in the respective receptacles 2715.

Embodiments of a surgical tool aimer 2701 can include a lock plate comprising the ratchet features. The lock plate can be located inside the handle 2711. The lock plate can have a tooth for each respective receptacle 2715. The lock plate can be located in a slot 2761 in the handle 2711 that is between columns of the receptacles 2715, such that the lock plate is mounted in the slot 2761.

Embodiments of the lock plate can be asymmetric. For example, the teeth for a first column of the receptacles 2715 can be smaller than the teeth for a second column of the receptacles 2715. Pairs of the teeth can extend from respective tabs. Each tab can be cantilevered from the lock plate. Each pair of the teeth can be perpendicular to the respective tab.

In another embodiment of aimer similar to the embodiments of FIGS. 30 and 31, the receptacles of the first column of the receptacles may aim the bullet at a single target location along the aimer arm that is not the tip or the elbow, such as a target slot or some other point near to the tip, and the receptacles of the second column of the receptacles will accordingly aim the bullet at the same single target location along the aimer arm.

In another embodiment of aimer similar to the embodiments of FIGS. 30 and 31, the aimer may include a lock plate that is integrated with the handle and that includes the ratchet features. Such a lock plate may have a tooth for each respective receptacle that is cantilevered about the longitudinal centerline of the handle.

Any of the embodiments disclosed herein may use features or components from any other embodiment, with or without any limitations. Still other embodiments can include one or more of the following items.

1. A surgical tool aimer, comprising:

a handle configured to support and aim a bullet that is configured to target a surgical site;

an aimer arm pivotally coupled to the handle, and the aimer arm has a distal end with a tip and an elbow adjacent to the tip; and

an actuator coupled to the handle for selectively indexing the aimer arm about a pivot such that the tip or the elbow can be manually adjusted to be aimed at the bullet while the bullet and handle are stationary.

2. The surgical tool aimer wherein the actuator comprises a pin that is manually actuated.

3. The surgical tool aimer wherein the actuator comprises magnets located on opposite sides of the handle, and the magnets engage a proximal end of the aimer arm, opposite the tip and elbow of the aimer arm, in indexed positions.

4. The surgical tool aimer wherein the pivot is external relative to the handle, and the handle does not comprise any side windows for the bullet.

5. The surgical tool aimer wherein the pivot is inside the handle, and a proximal end of the aimer arm extends into the handle for about half of a length of the handle.

6. The surgical tool aimer wherein the handle is curved and is not straight, and a proximal end of the aimer arm is inside the handle.

7. The surgical tool aimer wherein the bullet and the aimer arm are in co-planar alignment.

8. The surgical tool aimer wherein longitudinal centerlines of the bullet and the aimer arm are in co-planar alignment.

9. The surgical tool aimer wherein the aimer arm comprises a fiducial marker configured to assist in video-based surgical navigation.

10. The surgical tool aimer wherein the handle comprises receptacles, and the bullet is configured to be installed in any one of the receptacles to selectively vary an angle of inclination of the bullet toward the surgical site.

11. The surgical tool aimer wherein the receptacles comprise a single column of receptacles, and the bullet is co-planar with the aimer arm.

12. The surgical tool aimer wherein each receptacle in the handle comprises a plunger that is spring-actuated, each plunger has an edge configured to ratchet and hold the bullet in a respective receptacle, and the edge engages teeth on the bullet.

13. A surgical tool aimer, comprising:

a handle configured to support and aim a bullet that is configured to target a surgical site;

an aimer arm pivotally coupled to the handle at a pivot, and the aimer arm has a tip and an elbow adjacent to the tip; and

an actuator coupled to the handle for indexing the aimer arm about the pivot such that the tip or the elbow can be adjustably moved to be aimed at the bullet while the bullet and handle are stationary, and the actuator comprises a pin that is manually actuated.

14. The surgical tool aimer wherein the pivot is located inside or outside of the handle.

15. The surgical tool aimer wherein the handle is curved, and the bullet and the aimer arm are in co-planar alignment.

16. The surgical tool aimer wherein longitudinal centerlines of the bullet and the aimer arm are in co-planar alignment.

17. The surgical tool aimer wherein the aimer arm comprises a fiducial marker configured to assist in video-based surgical navigation; and

the handle comprises receptacles, and the bullet is configured to be installed in any one of the receptacles to selectively vary an angle of inclination of the bullet toward the surgical site.

18. The surgical tool aimer wherein the receptacles comprise a single column of receptacles, and the bullet is co-planar with the aimer arm.

19. A surgical tool aimer, comprising:

a handle configured to support and aim a bullet that is configured to target a surgical site;

an aimer arm pivotally coupled to the handle at a pivot, and the aimer arm has a tip and an elbow adjacent to the tip; and

an actuator coupled to the handle for selectively indexing the aimer arm about the pivot such that the tip or the elbow can be adjustably moved to be aimed at the bullet while the bullet and handle are stationary, and the actuator comprises magnets located on opposite sides of the handle, and the magnets engage a proximal end of the aimer arm, opposite the tip and elbow, in indexed positions.

20. The surgical tool aimer wherein the pivot is located inside or outside of the handle.

21. The surgical tool aimer wherein the handle is curved and the bullet and the aimer arm are in co-planar alignment.

22. The surgical tool aimer wherein longitudinal centerlines of the bullet and the aimer arm are in co-planar alignment.

23. The surgical tool aimer wherein the aimer arm comprises a fiducial marker configured to assist in video-based surgical navigation; and

the handle comprises receptacles, and the bullet is configured to be installed in any one of the receptacles to selectively vary an angle of inclination of the bullet toward the surgical site.

24. The surgical tool aimer wherein the receptacles comprise a single column of receptacles, and the bullet is co-planar with the aimer arm.

25. A surgical tool aimer, comprising:

a handle configured to support and aim a bullet that is configured to target a surgical site, the handle has receptacles for the bullet, and each of the receptacles is oriented at a unique orientation relative to the handle; and

an aimer arm rigidly mounted to the handle such that there is no relative motion therebetween, the aimer arm has a tip and an elbow adjacent to the tip, and the handle is configured to retain the bullet in one of the receptacles for selectively aiming the bullet at either the tip or the elbow.

26. The surgical tool aimer wherein the receptacles comprise a single column of receptacles, and the bullet is not co-planar with the aimer arm.

27. The surgical tool aimer wherein the receptacles comprise two columns of receptacles, and the bullet is not co-planar with the aimer arm such that the bullet is offset and skewed relative to the aimer arm.

28. The surgical tool aimer wherein a first column of the two columns of receptacles aims the bullet at the tip target, and a second column of the two columns of receptacles aims the bullet at the elbow target.

29. The surgical tool aimer further comprising a slot in the handle between the two columns of receptacles.

30. The surgical tool aimer wherein each receptacle comprises ratchet features configured to selectively engage and retain the bullet.

31. The surgical tool aimer wherein the handle comprises opposed spring clips comprising the ratchet features, each spring clip comprises a tooth for each respective receptacle, and the teeth are configured to engage and retain the bullet in the respective receptacles.

32. The surgical tool aimer further comprising a lock plate comprising the ratchet features, the lock plate is located inside the handle, and the lock plate has a tooth for each respective receptacle.

33. The surgical tool aimer wherein the handle further comprises a slot between columns of the receptacles, and the lock plate is mounted in the slot.

34. The surgical tool aimer wherein the lock plate is asymmetric, the teeth for a first column of the receptacles are smaller than the teeth for a second column of the receptacles, pairs of the teeth extend from respective tabs, each tab is cantilevered from the lock plate, and each pair of the teeth is perpendicular to the respective tab.

35. A surgical tool aimer, comprising:

a handle configured to support and aim a bullet that is configured to target a surgical site, the handle has receptacles for the bullet, and each of the receptacles is oriented at a unique orientation relative to the handle;

an aimer arm rigidly mounted and fixed relative to the handle for no relative motion therebetween, the aimer arm has a tip and an elbow adjacent to the tip, the handle is configured to retain the bullet in one of the receptacles for selectively aiming the bullet at either the tip or the elbow; and

the aimer arm and the bullet are laterally offset from a centerline of the handle, and the aimer arm and bullet are not co-planar such that the bullet is skewed relative to the aimer arm.

36. The surgical tool aimer wherein each receptacle comprises ratchet features configured to selectively engage and retain the bullet.

37. The surgical tool aimer wherein the handle comprises opposed spring clips comprising the ratchet features, each spring clip comprises a tooth for each respective receptacle, and the teeth are configured to engage and retain the bullet in the respective receptacles.

38. The surgical tool aimer further comprising a lock plate comprising the ratchet features, the lock plate is located inside the handle, and the lock plate has a tooth for each respective receptacle.

39. The surgical tool aimer wherein the handle further comprises a slot between columns of the receptacles, and the lock plate is mounted in the slot.

40. The surgical tool aimer wherein the lock plate is asymmetric, the teeth for a first column of the receptacles are smaller than the teeth for a second column of the receptacles, pairs of the teeth extend from respective tabs, each tab is cantilevered from the lock plate, and each pair of the teeth is perpendicular to the respective tab.

41. A surgical tool aimer, comprising:

a handle for supporting and aiming a bullet that is configured to target a surgical site, the handle has receptacles for the bullet, each of the receptacles is oriented at a unique orientation relative to the handle, and the handle is symmetrical;

an aimer arm rigidly mounted and not movable relative to the handle, the aimer arm has a tip and an elbow adjacent to the tip, the handle is configured to retain the bullet in one of the receptacles for selectively aiming the bullet at either the tip or the elbow, and the aimer arm is co-planar with a centerline of the handle;

the receptacles comprise first and second columns of receptacles, the first column of the receptacles aims the bullet at the tip, and the second column of the receptacles aims the bullet at the elbow.

42. The surgical tool aimer wherein the bullet is not co-planar with the aimer arm such that the bullet is skewed relative to the aimer arm.

43. The surgical tool aimer wherein the handle comprises a longitudinal slot outboard of each receptacle, and the longitudinal slots are configured to enable guidewire removal from respective ones of the receptacles.

44. The surgical tool aimer further comprising a lock plate comprising ratchet features, the lock plate is located inside the handle, and the lock plate has a tooth for each respective receptacle.

45. The surgical tool aimer wherein the handle further comprises a slot between columns of the receptacles, and the lock plate is mounted in the slot.

46. The surgical tool aimer wherein the lock plate is asymmetric, the teeth for a first column of the receptacles are smaller than the teeth for a second column of the receptacles, pairs of the teeth extend from respective tabs, each tab is cantilevered from the lock plate, and each pair of the teeth is perpendicular to the respective tab.

47. A surgical tool aimer, comprising:

a handle with bullet receptacle for supporting a bullet and aiming the bullet to target a surgical site, and the handle comprises aimer receptacles;

an aimer arm comprising metal and a laser-marked fiducial configured to assist in video-based surgical navigation, the aimer arm also comprises a tip and an elbow adjacent to the tip, the aimer arm is configured to be installed in any one of the aimer receptacles to selectively vary an angle of inclination of the aimer arm relative to the handle and the bullet, such that the bullet is selectively aimed at either the tip or the elbow of the aimer arm.

48. The surgical tool aimer wherein the handle is reusable and the aimer arm is disposable.

49. The surgical tool aimer wherein both the handle and the aimer arm are disposable, and the bullet is threaded into the handle to adjust a length of the bullet relative to the aimer arm.

50. The surgical tool aimer wherein the handle is arcuate in shape and the aimer arm and bullet are linear in shape, other than a distal tip of the aimer arm.

51. The surgical tool aimer wherein the aimer receptacles comprise a first array of aimer receptacles, and the handle further comprises a second array of aimer receptacles.

52. The surgical tool aimer wherein each aimer receptacle of the first array of aimer receptacles is configured to aim the bullet only at the tip, and each aimer receptacle of the second array of aimer receptacles is configured to aim the bullet only at the elbow.

53. The surgical tool aimer wherein the first array of aimer receptacles is located adjacent a proximal end of the handle, and the second array of aimer receptacles is located adjacent a distal end of the handle.

54. The surgical tool aimer wherein the handle consists of only one bullet receptacle that is the only location on the handle to which the bullet can be mounted.

55. The surgical tool aimer wherein the aimer arm can be mounted in any one of the aimer arm receptacles, but the aimer arm is not otherwise adjustable relative to the handle.

56. The surgical tool aimer wherein the handle comprises a side window for the bullet.

57. The surgical tool aimer wherein the aimer arm extends through a front of the handle, through one of the aimer receptacles, and is releasably secured to the handle with a fastener located on a rear of the handle.

58. The surgical tool aimer wherein a proximal end of the aimer arm is tapered, each of the aimer receptacles are complementary in shape to the proximal end of the aimer arm, and a distal end of the aimer arm comprises a fiducial.

59. The surgical tool aimer wherein the handle, bullet and aimer arm are in co-planar alignment.

60. The surgical tool aimer wherein longitudinal centerlines of the handle, bullet and aimer arm are in co-planar alignment.

61. A surgical tool aimer, comprising:

a handle for supporting and aiming a bullet that is configured to target a surgical site, the handle has receptacles for the bullet, each of the receptacles is oriented at a unique orientation relative to the handle, and the handle comprises a primary handle and a secondary handle;

an aimer arm mounted to the handle, the aimer arm has a tip and an elbow adjacent to the tip, and the handle is configured to retain the bullet in one of the receptacles for selectively aiming the bullet at at least one of the tip or the elbow; and

a lock plate mounted between the primary handle and the secondary handle, and the lock plate has a tooth for each respective receptacle in the handle, such that the teeth respectively engage bullet teeth on the bullet to retain the bullet in the handle.

62. The surgical tool aimer wherein both the primary handle and the secondary handle comprise receptacles.

63. The surgical tool aimer wherein both the primary handle comprises a first column of receptacles that aim the bullet at the tip, the secondary handle comprises a second column of receptacles that aim the bullet at the elbow.

64. The surgical tool aimer wherein the secondary handle is removable from the primary handle, and an alternative handle without receptacles is mounted to the primary handle, with the lock plate between the primary handle and the alternative handle.

65. The surgical tool aimer wherein each receptacle comprises a side window to an exterior of the handle.

66. The surgical tool aimer wherein the secondary handle slidably engages the primary handle to retain the lock plate inside the handle, and the handle comprises retention features to align and secure the secondary handle to the primary handle.

67. The surgical tool aimer wherein the retention features comprises one or more of:

a central rib on the primary handle that engages a central slot on the secondary handle; or

flanges on opposite sides of the primary handle that slidably engage complementary flanges, respectively, on opposite sides of the secondary handle; or

tabs on the primary handle that engage spring tabs on the secondary handle.

68. A surgical tool aimer, comprising:

a handle configured to support and aim a bullet that is configured to target a surgical site, the handle has receptacles for the bullet, wherein the receptacles comprise a first column of receptacles and a second column of receptacles, and each of the receptacles in the first column is oriented at a unique orientation relative to the handle, and each of the receptacles in the second column mirrors, about a longitudinal centerline of the handle, the orientation of a respective receptacle in the first column; and

an aimer arm rigidly mounted to the handle such that there is no relative motion therebetween, and the handle is configured to retain the bullet in one of the receptacles for selectively orienting the bullet for aiming at a single target location along the aimer arm,

wherein a longitudinal slot is outboard of each receptacle to enable guidewire removal from respective ones of the receptacles.

69. The surgical tool aimer wherein the aimer arm comprises a tip, and the single target location is the tip.

70. The surgical tool aimer wherein the aimer arm comprises an elbow, and the single target location is the elbow.

71. The surgical tool aimer wherein the single target location is a point near to a tip of the aimer arm.

72. The surgical tool aimer wherein the bullet is not co-planar with the aimer arm such that the bullet is offset and skewed relative to the aimer arm.

73. The surgical tool aimer further comprising a slot in the handle between the two columns of receptacles.

74. The surgical tool aimer wherein each receptacle comprises ratchet features configured to selectively engage and retain the bullet.

75. The surgical tool aimer wherein the handle comprises opposed spring clips comprising the ratchet features, each spring clip comprises a tooth for each respective receptacle, and the teeth are configured to engage and retain the bullet in the respective receptacles.

76. The surgical tool aimer further comprising a lock plate comprising the ratchet features, the lock plate is inside the handle, and the lock plate has a tooth for each respective receptacle.

77. The surgical tool aimer wherein the handle further comprises a slot between columns of the receptacles, and the lock plate is mounted in the slot.

78. The surgical tool aimer wherein the lock plate is asymmetric, the teeth for a first column of the receptacles are smaller than the teeth for a second column of the receptacles, pairs of the teeth extend from respective tabs, each tab is cantilevered from the lock plate, and each pair of the teeth is perpendicular to the respective tab.

79. The surgical tool aimer further comprising a lock plate comprising the ratchet features, the lock plate integrated with the handle, and the lock plate has a tooth for each respective receptacle that is cantilevered about the longitudinal centerline of the handle.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” “top”, “bottom,” and the like, may be used herein for ease of description to describe one element’s or feature’s relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated degrees or at other orientations) and the spatially relative descriptions used herein interpreted accordingly.

This written description uses examples to disclose the embodiments, including the best mode, and also to enable those of ordinary skill in the art to make and use the invention. The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

It can be advantageous to set forth definitions of certain words and phrases used throughout this patent document. The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, C, A and B, A and C, B and C, and A and B and C.

Also, the use of “a” or “an” is employed to describe elements and components described herein. This is done merely for convenience and to give a general sense of the scope of the invention. This description should be read to include one or at least one and the singular also includes the plural unless it states otherwise.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims.

After reading the specification, skilled artisans will appreciate that certain features that are, for clarity, described herein in the context of separate embodiments, can also be provided in combination in a single embodiment. Conversely, various features that are, for brevity, described in the context of a single embodiment, can also be provided separately or in any sub-combination. Further, references to values stated in ranges include each and every value within that range.

What is claimed is:

1. A surgical tool aimer, comprising:
a handle configured to support and aim a bullet that is configured to target a surgical site, the handle has receptacles for the bullet, and each of the receptacles is oriented at a unique orientation relative to the handle; and
an aimer arm rigidly mounted to the handle such that there is no relative motion therebetween, the aimer arm has a tip and an elbow adjacent to the tip, and the handle is configured to retain the bullet in one of the receptacles for selectively aiming the bullet at either the tip or the elbow.

2. The surgical tool aimer of claim 1, wherein the receptacles comprise a single column of receptacles, and the bullet is not co-planar with the aimer arm.

3. The surgical tool aimer of claim 1, wherein the receptacles comprise two columns of receptacles, and the bullet is not co-planar with the aimer arm such that the bullet is offset and skewed relative to the aimer arm.

4. The surgical tool aimer of claim 3, wherein a first column of the two columns of receptacles aims the bullet at the tip, and a second column of the two columns of receptacles aims the bullet at the elbow.

5. The surgical tool aimer of claim 1, wherein each receptacle comprises ratchet features configured to selectively engage and retain the bullet.

6. The surgical tool aimer of claim 5, wherein the handle comprises opposed spring clips comprising the ratchet features, each spring clip comprises a tooth for each respective receptacle, and the teeth are configured to engage and retain the bullet in the respective receptacles.

7. The surgical tool aimer of claim 6, further comprising a lock plate comprising the ratchet features, the lock plate is located inside the handle, and the lock plate has a tooth for each respective receptacle.

8. The surgical tool aimer of claim 7, wherein the lock plate is asymmetric, the teeth for a first column of the receptacles are smaller than the teeth for a second column of the receptacles, pairs of the teeth extend from respective tabs, each tab is cantilevered from the lock plate, and each pair of the teeth is perpendicular to the respective tab.

9. A surgical tool aimer, comprising:
a handle for supporting and aiming a bullet that is configured to target a surgical site, the handle has receptacles for the bullet, each of the receptacles is oriented at a unique orientation relative to the handle, and the handle is symmetrical;
an aimer arm rigidly mounted and not movable relative to the handle, the aimer arm has a tip and an elbow adjacent to the tip, the handle is configured to retain the bullet in one of the receptacles for selectively aiming the bullet at either the tip or the elbow, and the aimer arm is co-planar with a centerline of the handle; and
the receptacles comprise first and second columns of receptacles, the first column of the receptacles aims the bullet at the tip, and the second column of the receptacles aims the bullet at the elbow.

10. The surgical tool aimer of claim 9, wherein the bullet is not co-planar with the aimer arm such that the bullet is skewed relative to the aimer arm.

11. The surgical tool aimer of claim 9, wherein the handle comprises a longitudinal slot outboard of each receptacle, and the longitudinal slots are configured to enable guidewire removal from respective ones of the receptacles.

12. The surgical tool aimer of claim 9, further comprising a lock plate comprising ratchet features, the lock plate is located inside the handle, and the lock plate has a tooth for each respective receptacle.

13. The surgical tool aimer of claim 12, wherein the lock plate is asymmetric, the teeth for a first column of the receptacles are smaller than the teeth for a second column of the receptacles, pairs of the teeth extend from respective tabs, each tab is cantilevered from the lock plate, and each pair of the teeth is perpendicular to the respective tab.

14. A surgical tool aimer, comprising:
a handle configured to support and aim a bullet that is configured to target a surgical site, the handle has receptacles for the bullet, wherein the receptacles comprise a first column of receptacles and a second column of receptacles, and each of the receptacles in the first column is oriented at a unique orientation relative to the handle, and each of the receptacles in the second column mirrors, about a longitudinal centerline of the handle, the orientation of a respective receptacle in the first column; and
an aimer arm rigidly mounted to the handle such that there is no relative motion therebetween, and the handle is configured to retain the bullet in one of the receptacles for selectively orienting the bullet for aiming at a single target location along the aimer arm,
wherein a longitudinal slot is outboard of each receptacle to enable guidewire removal from respective ones of the receptacles.

15. The surgical tool aimer of claim 14, wherein the aimer arm comprises a tip, and the single target location is the tip.

16. The surgical tool aimer of claim 14, wherein the aimer arm comprises an elbow, and the single target location is the elbow.

17. The surgical tool aimer of claim 14, wherein the single target location is a point near to a tip of the aimer arm.

18. The surgical tool aimer of claim 14, wherein the bullet is not co-planar with the aimer arm such that the bullet is offset and skewed relative to the aimer arm.

19. The surgical tool aimer of claim 14, wherein each receptacle comprises ratchet features configured to selectively engage and retain the bullet.

20. The surgical tool aimer of claim 19, wherein the handle comprises opposed spring clips comprising the ratchet features, each spring clip comprises a tooth for each respective receptacle, and the teeth are configured to engage and retain the bullet in the respective receptacles.
